(19) United States
(12) Patent Application Publication
(10) Pub. No.: US 2008/0262612 A1
(43) Pub. Date: Oct. 23, 2008

(54) Bio Artificial Eye and Conformer

(76) Inventors: Mal-Soo Jun, Busan (KR); Jong-Heon Jun, Busan (KR); Seong-Un Jun, Busan (KR); Yu-Mi Jun, Busan (KR)

Correspondence Address: IPLA P.A., 3580 WILSHIRE BLVD., 17TH FLOOR, LOS ANGELES, CA, 90010, US

(21) Appl. No.: 11/816,694

(22) PCT Filed: Jul. 7, 2005
(86) PCT No.: PCT/KR2005/002151

§ 371 Date: Aug. 20, 2007

(30) Foreign Application Priority Data
Mar. 2, 2005 (KR) 10-2005-0017153
Mar. 16, 2005 (KR) 10-2005-0022525
Mar. 16, 2005 (KR) 10-2005-0022527
May 24, 2005 (KR) 10-2005-0044325
May 24, 2005 (KR) 10-2005-0044327

(51) Int. Cl. A61F 2/14

(52) U.S. Cl. 623/6.64

(57) ABSTRACT

The present invention relates to a bio-artificial eye which is produced by a process comprising adding loess, zeolite, bentonite, bioceramic or nano-silver to acrylic resin powder. Thus, the artificial eye has antibacterial activity in itself, does not cause much eye discharge, and emits far-infrared rays and anions, and thus, has a very good effect on the ocular health of a person wearing the artificial eye. Furthermore, the present invention relates to a conformer which is produced by a process comprising adding loess, zeolite, bentonite, bioceramic or nano-silver to acrylic resin powder. Thus, the inventive conformer has antibacterial activity in itself, does not cause much eye discharge, and emits far-infrared rays and anions, and thus, has a very good effect on the ocular health of a person wearing the conformer.

### TECHNICAL FIELD

Generally, an artificial eye is a pseudo eyeball which is worn to provide a normal appearance when it is impossible for one eye to recover visual functionality due to ocular disorders or eyeball injury. The artificial eye functions as an artificial eyeball necessary for a person blind in one eye to live a harmonious social life without mental lack and care for the eyes of other people.

The present invention relates to an artificial eye and a conformer which is temporarily used by insertion into an eye socket after eyeball surgery. More particularly, the present invention relates to a bio-artificial eye and a bio-conformer, which are produced by a process comprising adding loess, zeolite, bentonite, bioceramic, nano-silver, nano-gold or nano-platinum to acrylic resin powder or silicone, so that the artificial eye and the conformer have antibacterial activity in themselves, do not cause much eye discharge, and emit far-infrared rays and anions, and thus, have a very good effect on the ocular health of a person wearing the artificial eye.

### BACKGROUND ART

Generally, the artificial eye is a kind of artificial eyeball which is produced in a shape like a healthy eye and worn by a person blind in one or both eyes due to injury or congenital factors, thus causing a third person to feel no revulsion. The artificial eye requires that a person feel comfortable during the wearing of the artificial eye so that the person can lead a pleasant everyday life.

Also, the artificial eye is used for persons who have undergone eyeball enucleation due to injury or lesion in an eye or its surrounding area, persons who have no eyesight due to the postnatal contraction or underdevelopment of an eyeball, and persons who cannot be expected to recover their sight. It is used to protect the eye orbit, maintain the formation of the eye orbit and promote the development of bone structure. In addition, it is used to give a better appearance, like a normal eye, thus giving a wearer self-confidence.

FIG. 1 is a hypothetical side view showing a state before an artificial eye is inserted into the eye of a patient. As shown in FIG. 1, the eye 11 consists of the cornea 16, iris 18, crystalline lens, visual nerve, and the like. The cornea 16, which is tissue having no transparent blood vessels 19 at the outermost portion of the eye 11, is frequently called a “black pupil”, and functions as a protective membrane for protecting an eyeball and as a window for allowing light to reach the retina by refraction. The iris 18 is located between the cornea 16 and the crystalline lens, and its color can vary depending on human species and individuals but is colored brown by much pigment and blue by less pigment. Also, it functions to control the amount of light. The crystalline lens, which is a colorless transparent structure having the shape of a magnifying glass convex on both sides, is located behind the iris and acts as the main refractive organ together with the corneal 16.

When an eyeball is enucleated from the eye having this structure, an implant 12 as an orbit filler will be inserted into the eye, and an artificial eye (ocular prosthesis) 10 will be inserted into the conjunctival sac 13 of the orbit where the cornea 16 and the iris 18 were located. When an eyeball is contracted or underdeveloped, only the artificial eye 10 will be inserted into the conjunctival sac 13 of the orbit.

As described above, the artificial eye 10 should be inserted into the conjunctival sac 13 of the orbit so as to make close contact with the eyeball. For this reason, it should be formed so as to conform to the shape of the conjunctival sac 13 of each individual, and the shape of its pupil should be the same as the actual eye of a normal person. When a person is blind in only one eye, the artificial eye should be formed as similarly as possible to the other normal eye of the person in order to improve wearing comfort and to minimize heterogeneity in appearance.

A general method for producing the artificial eye 10 is as follows.

First, the conjunctival sac of the orbit, which is to be inserted with the artificial eye, is injected with a molding material by the use of a device for modeling the artificial eye 10, thus making a model of the artificial eye. In this regard, the molding material is a material, such as alginate which is mainly used to make tooth models in dentistry.

The artificial eye model thus prepared is placed in a two-part flask to make a moulage of the artificial eye 10. Meanwhile, transparent or non-transparent high-purity polymethylmethacrylate (PMMA) resin powder and PMMA resin solution are polymerized at a ratio of about 2.2:1 and gelled at room temperature for about 15-20 minutes. The polymer gel is placed in the moulage, compressed with a press, and cured by heating at 71-74° C. for about 1 hour and 30 minutes followed by heating at 100° C. for 30 minutes, thus making an artificial eye body to be inserted into an orbit.

The entire surface of the artificial eye body is subjected to a cutting process, and then, an eye pupil and blood vessels 19 of the eye white are drawn on the surface of the artificial eye body. Meanwhile, polymethylmethacrylate(PMMA) resin powder and PMMA resin liquid are polymerized at a ratio of about 2.2:1 and gelled at room temperature for about 15-20 minutes. Then, the artificial eye body is placed in a press and covered with the polymer gel. The resulting artificial eye body is compressed, cured with heat, cut and polished, thus making an artificial eye.

The iris, which is the central part of an artificial eye, is characterized depending an each individual, has a very elaborate shape and color, and determines personal appearance.

Also, the applicant has developed an artificial eye 10 having tear holes 50 formed in the conjunctiva 20. The developed artificial eye has the effect that it can be used for a long time due to the supply of a large amount of oxygen and the smooth circulation of tears compared to an artificial eye having no tear holes.

Korean utility model registration No. 20-0329880 discloses a soft artificial eye made of silicone in the prior art. The soft artificial eye according to the prior art is an artificial eye 10 comprising an artificial pupil, an iris 18 and a sclera 17 having blood vessels drawn thereon, in which the sclera 17 is formed of a silicone rubber material so as to have softness.

In another embodiment, the soft artificial eye further comprises a transparent coating layer which is deposited on the sclera 17 so as to cover the pupil, the iris 18 and the blood vessels. Another soft artificial eye made of silicone is disclosed in Korean utility model laid-open publication No. 1998-56780. The outer surface of this artificial eye 10 is adsorbed with hydroxy apatite powder which has a chemical and physical structure similar to the bone of the human body, and thus, does not cause side effects in the human body. Due to the hydroxy apatite powder, the blood vessels and tissue of a patient propagate on the artificial eye 10 and form one living portion of the orbit so as to make the free movement of the artificial eye possible.

However, all the artificial eyes 10 according to the prior art have problems in that bacteria propagate on the orbit and the artificial eye 10 so that eye discharge is easily caused and inflammation occurs.

One which is temporarily used by insertion into an eye region after eyeball surgery but before the wearing of the artificial eye 10 is called a “conformer” 100. More specifically, the conformer 100 has tear holes 400 formed in an elliptical or circular shape which is temporarily used by insertion into the eye region for the purpose of preventing an eyelid from adhering to a surgical site after surgery and for the purpose of eliminating a foreign body sensation caused by a surgical seam. Usually, the conformer 100 is replaced with the artificial eye 10 4-6 weeks after its insertion into the eye.

A general method for producing the conformer 100 is as follows.

The model of the conformer 100 is made with paraffin wax, and the conformer model is placed in a two-part flask to make a moulage of the conformer 100. Meanwhile, transparent or non-transparent polymethylmethacrylate (PMMA) resin powder and PMMA resin solution are mixed with each other and gelled. The polymer gel is injected into the moulage and cured, thus making a conformer body to be inserted into an orbit.

However, all the conformers 100 according to the prior art have problems in that bacteria propagate on the orbit and the conformer 100 as in the prior artificial eye so that eye discharge is easily generated and inflammation occurs.

### DISCLOSURE

### Technical Problem

The present invention has been made to solve the above-described problems occurring in the prior art, and it is an object to provide an artificial eye which contains materials having antibacterial and bactericidal activities and emitting far-infrared rays and anions, in addition to the material of the prior artificial eye, so that the artificial eye and an orbit can always be maintained in a hygienic condition, and the cells of the orbit region can be activated by far-infrared rays and the like to maintain a healthy state.

Another object of the present invention is to provide a conformer which contains materials having antibacterial and bactericidal activities and emitting far-infrared rays and anions, in addition to the material of the prior conformer, so that the conformer and an orbit can always be maintained in a hygienically good condition, and the cells of the orbit region can be activated by far-infrared rays and the like to maintain a healthy state.

### Technical Solution

The present invention relates to a bio-artificial eye and a conformer.

In one embodiment, the present invention provides a bio-artificial eye comprising a polymer of polymethylmethacrylate (PMMA) resin powder and PMMA liquid resin, and any one selected from the group consisting of loess, bentonite, zeolite, bioceramic, nano-silver, nano-gold and nano-platinum.

In another embodiment, the inventive bio-artificial eye comprises silicone and any one selected from loess, bentonite, zeolite, bioceramic, nano-silver, nano-gold and nano-platinum.

In the inventive bio-artificial eye, the content of any one of the loess, bentonite and bioceramic components is less than 30% by weight, and the remaining component is the polymer of transparent or non-transparent polymethylmethacrylate (PMMA) resin powder and PMMA resin liquid.

In the inventive bio-artificial eye, the content of the nano-silver component is 0.01-5% by weight, and the remaining component is the polymer of transparent or non-transparent polymethylmethacrylate (PMMA) resin powder and PMMA resin liquid.

In the inventive bio-artificial eye, the content of any one of the nano-silver and nano-platinum components is 0.01-5% by weight, and the remaining component is the polymer of transparent or non-transparent polymethylmethacrylate (PMMA) resin powder and PMMA resin liquid.

In the inventive bio-artificial eye, the content of any one of the loess, bentonite, zeolite and bio-ceramic components is less than 30% by weight, and the remaining component is silicone.

In the inventive bio-artificial eye, the content of any one of nono-silver, nano-gold and nano-platinum is 0.01-5% by weight, and the remaining component is silicone.

The loess used in the present invention is a mass of very fine sand and contains a large amount of calcium carbonate, and thus, is not brittle. Because of this calcium carbonate, the loess is changed to clay when mixed with water.

The loess has a particle size of 0.02-0.05 mm and consists of various mineral particles, such as quartz, feldspar, mica, calcite and the like. The chemical composition of the loess comprises 50-60% SiO₂, 20-30% Al₂O₃, 5-10% Fe₂O₃, and 5-10% others (MgO, CaO, K₂O, CuO, ZnO and TiO₂).

Meanwhile, loess is frequently called a “living organism” because a spoon of loess contains about 200,000,000 microorganisms. This fact that loess is living organism, demonstrates that loess has a great effect on the human beings.

Microorganisms, such as moss, mold and bacteria, which are contained in the loess, function to decompose organic substances, in which the organic substances decomposed by these microorganisms become a nutrient source for plants and are also used as drugs for treating human diseases.

Furthermore, loess contains various enzymes performing circulation actions, the explanation of several of which can provide the understanding of the various effects of the loess on human beings.

First, loess, covering about 10% of the earth's surface, has a large amount of calcium carbonate (CaCO₃) by which the loess is viscous so as not to be brittle and has the property of changing to clay upon the addition of water. Also, loess consists of silica (SiO₂), alumina (Al₂O₃), iron, magnesium (Mg), sodium (Na), potassium and the like. Because loess consists of these mineral components and various enzymes, it emits a large amount of far-infrared rays necessary for animals and plants. For this reason, loess is called “living organism”.

Loess has a honeycomb structure with large surfaces, in which numerous spaces form a multilayer structure, and a large amount of far-infrared rays are absorbed and stored in the sponge-like holes and emitted upon the application of heat to stimulate the molecular activity of other objects. Loess is a silicon-based mineral which has absorbed solar energy for a long period of time, and can be said to be a solar energy reservoir.

Loess in Korea consists of yellow sand blowing from the continent of China, and the loess contains four kinds of enzymes, catalase, diphenol oxidase, saccharase and protease. These enzymes perform the actions of toxin removal, decomposition, fertilizer urea, and purification, respectively. Catalase, an enzyme of loess, shows the highest activity among the soil enzymes and functions to remove hydrogen peroxide toxic to organisms, thus making the soil environment suitable for living organisms.

The development of lipid peroxide, a toxin, during the metabolic process in the human body, leads to an aging phenomenon. When the body is placed in good quality loess, lipid peroxide as a toxin of the body can be neutralized and diluted due to the strong absorption activity of the loess. Thus, the loess inhibits human aging and maintains youth.

The second enzyme of the loess is diphenol oxidase which acts to make the oxidation and decomposition activities of the loess strong.

The third enzyme of loess is saccharase, and comparative tests for loess and black earth showed that the loess is higher in saccharase activity than black earth. The use of loess in rice or barley cultivation offered a comparable result with urea in fertilizer. This is considered to have a close connection with the fact that rice and barley cultivation is prevalent in the south region of Korea, which is rich in loess.

The fourth enzyme of loess is protease which hydrolyzes protein into amino acids when the nitrogen of protein becomes inorganic. Namely, this enzyme functions to purify soil by a kind of decomposition action, as nitrogen contained in the protein of a dead animal body is converted into amino acids. Thus, for living organisms, cancer, tumors or other decayed cells can be decomposed with the aid of protease, an enzyme contained in the soil. This is supported by the fact that folk remedies are utilized to treat festering wounds or to remove toxins from the body.

In addition, various enzymes contained in loess are active by themselves, and as a result, have advantageous effects on human beings.

To summarize the effects of loess, loess promotes blood circulation, makes the metabolism active, and has good effects against arthritis, muscular pain, lumbago, and autonomic imbalance (traffic accident sequela). Also, it has a good effect on skin beauty due to its abilities to decompose and purify body waste materials, and relieves pain by removing body toxins. In addition, it has the effects of inhibiting cancer by alleviating inflammation and preserving a tranquil mind to strengthen the body and mind.

Zeoilte used in the inventive artificial eye is an aluminum silicate mineral which contains alkali metals, such as sodium and potassium, alkaline earth metals, such as calcium, and water of crystallization, and its kinds are very large, but two kinds, clinoptilolite and mordenite, are generally distributed.

Natural zeolite, which occurs as rock or clay, results from the modification of tuff, and thus, has strong adsorption, absorption and storage actions with base exchange capacity, although these properties are inferior to synthetic zeolite. The exchange action indicates that zeolite is ionized in water so that it can be exchanged with other electrolyte ions. All arsenic groups are rich in needle, column, rhombohedron and bob crystals, and are colorless or have white or light colors. Also, they have a specific gravity of 2-2.4 (2.7) and a hardness of 3.5-5.5, are soluble at low temperature, and easily discharge water upon heating, but arsenic water is present therein.

Natural zeolite is used a stock feed additive to promote the growth of livestock, and is added to soil to increase crop yield, and is used to manufacture building bricks so as to maintain indoor temperature and humidity at constant levels in all seasons.

Zeolite is a three-dimensional structure consisting of Al2O3 and SiO2 tetrahedrons, has pores and channels in the structure and contains exchangeable metal ions. Because of this structural property, it can be used in various applications, such as water treatment agents, molecular sieves, catalysts, catalyst supports, and ceramic additives.

Bentonite used in the inventive artificial eye is a mineral produced by events where volcanic ash and molten rock caused by volcanic eruption in the earth's formation period move onto land by crustal movements after sedimentation in the sea for a long period of time. It is also called “montmorillonite”, which is the broad name of bentonite.

Bentonite (montmorillonite) has been classified by the FDA as GRAS, and it has the effects of aiding food digestion and treating various diseases (e.g., allergies, paradentitis, streptococci of the gullet, gastric ulcers, vomiting, anal and rectal bleeding, diarrhea, wasp stings, water eczema, acnes, boils, women's yeast infection, child fever, and itching). Also, it has the effects of cleaning blood, reducing the stress of the nerve system, promoting metabolism and enhancing vitality. In addition, it shows excellent effects on skin beauty and treatment.

(1) Eco-Friendly, Safe and Stable Mineral

Because bentonite is an inorganic material, it is not spoiled, unlike an organic material, and has high thermal resistance, so that it is stable up to about 70° C.

(2) Swellability

Bentonite has a high affinity for water and when in contact with water, will absorb water to swell several to several tens of times its original volume, like a jelly phase. Also, it stores absorbed water by itself and prevents absorbed water from flowing out.

(3) Absorption Ability

Bentonite can absorb not only water but also oil, dirt and odors.

(4) Sol/Gel Formation Ability

Bentonite can form sol even with the addition of a small amount of water, and when this gel is left to stand, it becomes hardened like gel.

(5) Protection by Formation of Thin Film

Bentonite liquid has the property of easily forming a smooth film.

Silver used in the inventive artificial eye is the most powerful natural antibacterial agent.

Silver is a catalyst that interacts with a peculiar enzyme with which monads such as bacteria, virus, and fungi perform their respiration, digestive process and metabolism, and incapacitates the monads to suffocate them or starve them to death. The protoplasm of most germs is destroyed or their reproductive organs for cell division are melted by the electrical impact of silver ion (Ag). Physicians say that the drinking of silver ion water shows effects against syphilis, cholera, malaria, cold, water eczema, and severe scalds.

It is experimentally proven that metal silver can kill almost all unicellular germs present on the ground. The fact that there is no germ surviving for more than 6 minutes when in contact with silver dramatically indicates the powerful antibacterial function of silver.

In addition to killing germs when in a metal state, silver will remove the reproductive function of bacteria in water when it is discharged into water in an ionized state (Ag+). Thus, silver has the bactericidal function of killing germs and the antibacterial function to prevent the propagation of germs.

Nano-silver used in the present invention, which is a compound word of nono-technology and silver, consists of powder and solution. Nano-silver is made in view of excellent effects, such as antibacterial activity, deodorizing activity and the extension of food preservation time, among the various properties of silver.

Hereinafter, the present invention will be described in detail with reference to the accompanying drawings.

FIG. 1 is a hypothetical side view showing a state before an artificial eye is inserted into the eye of a patient, FIG. 2 is a detailed front view of the prior artificial eye, FIG. 3 shows the prior artificial eye having tear holes formed therein, FIG. 4 is a photograph showing the back side of the inventive artificial eye containing 15% by weight loess, and FIG. 5 is a photograph showing the inventive artificial eye containing 0.2% by weight nano-silver.

As shown in FIGS. 2 and 3, the artificial eye 10 comprises a central pupil portion 30, an iris portion 40 surrounding the pupil portion 40, and a conjunctiva portion 40 outside the iris portion 40, in which tear holes are formed in the conjunctiva portion 40.

Although loess used in the present invention preferably has a particle size of 1000-3000 meshes, loess with various particle sizes, if necessary, is added to make an artificial eye.

Zeolite and bentonite having a particle sizes of about 1,000-3,000 meshes are used to produce an artificial eye. Also, bioceramic particles useful to the human body, such as jade, germanium and bio-stone, may be added to produce an artificial eye. Also, nano-silver particles may be added to produce an artificial eye.

In another embodiment, nano-gold or nano-platinum may be used to produce an artificial eye. In this case, the production method is the same as the method with the use of nano-silver, and nano-gold or nano-platinum is most preferably used in an amount of 0.01-5% by weight, because the use of too much nano-gold or nano-platinum results in a deep color, and the use of too little nano-gold or nano-platinum shows little or no effect and decreases the beauty of an artificial eye.

However, the use of more than 0.001% by weight of nano-gold or nano-platinum can achieve the minimum desired effect, and its content can also vary according to the demand and request of a user.

For reference, nano-gold or nano-platinum, which is mainly used in the present invention, will now be described.

Gold is excellent in antibacterial activity, anti-mold activity, sterilizing and deodorizing effects, electrical conductivity and far-infrared emission, so that it is applied to human body-friendly goods and is used for the study of various industrial materials. Also, it performs nerve-stabilizing action, detoxifying action, ionic action and an action of making blood circulation smooth. Furthermore, gold generally has the effects of preserving a tranquil mind, stabilizing the mind and removing toxins. In addition, it is harmless to the human body, has no resistance to eco-friendly viruses, and shows no oxidation events.

Nano-gold is produced in the form of nano-particles, with a size of 1-30 nm, or a solution, and is brown or wine in color.

Platinum is superior in antibacterial effect to any synthetic drugs, and particularly, the advantage of platinum nano-technology is that the volume versus weight of platinum rapidly increases and the energy of each unit is generated on the same area with the same effect. For example, the treatment of 1 g of platinum by nano-technology generates a volume capable of drawing a line with a length of 180 million kilometers.

Also, the greatest advantage of nano-metal is that it has electrical properties and an antibacterial effect superior to any organic antibacterial drugs. The results of study by FITI Testing & Research Institute demonstrated that when each of gold, silver and platinum nano-particles having a size of 20 nm was added to water containing 140,000/ml of bacteria at a concentration of 10 ppm, each of the nano-particles showed an antibacterial activity of more than 99.9%, and among these nano-particles, nano-platinum showed superior antibacterial and deodorizing effects to any synthetic drugs.

Nano-platinum is produced in the form of nano-particles, with a size of 1-30 nm, or a solution, and is dark or khaki in color.

In another embodiment, the present invention relates to a bio-conformer, and more particularly to a conformer which is temporarily used by insertion into an eye socket after eyeball surgery. The inventive conformer comprises a polymer of transparent or non-transparent polymethylmethacrylate (PMMA) resin powder and PMMA resin liquid, and any one selected from the group consisting of loess, bentonite, zeolite, bio-ceramic material, nano-silver, nano-gold, and nano-platinum.

In the inventive conformer, the content of any one of loess, bentonite, zeolite and bioceramic is less than 30% by weight, and the remaining component is a polymer of transparent or non-transparent polymethylmethacrylate (PMMA) resin powder and PMMA resin liquid.

In the inventive conformer, the content of any one of nano-silver, nano-gold and nano-platinum is less than 0.01-5% by weight, and the remaining component is a polymer of transparent or non-transparent polymethylmethacrylate (PMMA) resin powder and PMMA resin liquid.

In another embodiment, the present invention provides a bio-conformer comprising silicone and any one component selected from the group consisting of loess, bentonite, zeolite, bio-ceramic, nano-silver, nano-gold, and nano-platinum.

Hereinafter, the conformer according to the present invention will be described in detail with reference to the accompanying drawings.

FIG. 6 shows the inventive bio-conformer, FIG. 7 is a photograph showing a conformer containing 5% by weight loess, and FIG. 8 is a photograph showing a conformer containing 20% by weight loess.

FIG. 9 is a photograph showing the inventive conformer containing 10% bioceramic, FIG. 10 is a photograph showing the inventive conformer containing 10% zeolite, and FIG. 11 is a photograph showing the inventive conformer containing 10% bentonite.

As shown in FIGS. 6 and 7, the conformer 100 comprises a central pupil portion 300 and a conjunctiva portion 200 surrounding the pupil portion 300, in which the conjunctiva portion 200 has one or two tear holes.

Although loess used in the present invention preferably a particle size of 1,000-3,000 meshes, loess having various particle sizes, if necessary, may be added to produce a conformer.

Zeolite or bentonite having a particle size of 1,000-3,000 meshes may be added to produce a conformer, and bio-ceramic particles useful to the human body, such as jade, germanium and bio-stone, may be added to produce a conformer.

In one embodiment of producing a conformer with the addition of loess, the loess is mixed with transparent or non-transparent polymethylmethacrylate (PMMA) resin powder, and the mixed powder is mixed with PMMA resin liquid and gelled at room temperature.

As in the mixing ratio, 1 g of loess is mixed with 10 g of the polymethylmethacrylate (PMMA) resin powder and then with 5 g of the PMMA resin solution. The mixing ratio can be adjusted according to the condition or request of a patient, and the acrylate resin powder can be used as a transparent or non-transparent material as required.

When the content of loess is low, the desired effect will be insignificant, and when loess is used in an amount of more than 30% by weight, it will result in a deep color and decrease the beauty of the conformer.

The gelled mixture is placed and compressed in a plaster mold, and heated at 71-74° C. for 1 hour and 30 minutes and then at 100° C. for 30 minutes, thus making a conformer.

In another embodiment, one corresponding to a portion of a conformer is prepared with the addition of loess, bentonite, zeolite, nano-silver, or bioceramic, and then, compressed with the remaining conformer portion prepared without the addition of the above component, thus making one complete conformer.

In this case, a portion of the conformer, which is prepared with the addition of the above component, preferably forms the back side of the conformer in view of beauty when a person wears the conformer.

As a result, in producing the conformer with the addition of loess, bentonite, zeolite, nano-silver or bio-ceramic material, (1) this component may be added throughout the conformer, and (2) a portion of the conformer prepared with the addition of this component may be compressed together with the remaining conformer portion with no addition of this component.

When nano-silver is contained in the conformer, it is preferably used in an amount of 0.01-5% by weight. This is because when too much nano-silver is used, it will result in a deep color, and when too little is used, it will have little or no effect and will injure the beauty of the conformer.

However, at least 0.001% by weight of nano-silver must be added in order to achieve the desired effect, and the amount used may vary according to the demand and request of a user.

Nano-silver powder or liquid is added in an amount of 0.01-5% by weight to plastic resin, acrylic resin, rubber or silicone, which is the material of the conformer.

A production process thereof is the same as the above-described process of producing the conformer with the addition of loess.

In another embodiment, a conformer is previously prepared, and then, in the final step, one or more selected from loess, bentonite, zeolite, nano-silver and bioceramic material is coated or applied on the outer surface of the prepared conformer.

In still another embodiment, nano-gold or nano-platinum can be used to produce a conformer, in which case a method of producing the conformer with the addition of nano-gold or nano-platinum is the same as the production method with the addition of nano-silver.

When nano-gold or nano-platinum is added to produce a conformer, it is most preferably used in an amount of 0.01-5% by weight for the same reason as in the case of adding nano-silver. 0.001% by weight of nano-gold or nano-platinum must be added in order to achieve the minimum desired effect, and the amount used can vary depending on the desire of a user.

In still another embodiment, a conformer is previously prepared with plastic resin, acrylic resin, rubber or silicone, and then, in the final stage, one or more selected from loess, bentonite, zeolite, nano-silver and bio-ceramic material is coated or applied on the outer surface of the prepared conformer.

### ADVANTAGEOUS EFFECTS

According to the present invention, a bio-artificial eye is produced with the addition of loess, zeolite, bentonite, bioceramic, nano-silver, or nano-platinum. Thus, the inventive artificial eye has bactericidal or antibacterial activity in itself, does not cause much eye discharge, and emits far-infrared rays and anions, and thus, has a very good effect on the ocular health of a person wearing the artificial eye.

Furthermore, according to the present invention, a conformer is produced with the addition of loess, zeolite, bentonite, bioceramic, nano-silver, or nano-platinum. Thus, the inventive artificial eye has bactericidal or antibacterial activity in itself, does not cause much eye discharge, and emits far-infrared rays and anions, and thus, has a very good effect on the ocular health of a person wearing the conformer.

### DESCRIPTION OF DRAWINGS

FIG. 1 is a hypothetical side view showing a state before an artificial eye is inserted into the eye of a patient.

FIG. 2 is a detailed front view of the prior artificial eye.

FIG. 3 shows the prior artificial eye having tear holes formed therein.

FIG. 4 is a photograph showing the back side of the inventive artificial eye containing 15% by weight loess.

FIG. 5 is a photograph showing the inventive artificial eye containing 0.2% by weight nano-silver.

FIG. 6 shows the inventive bio-conformer.

FIG. 7 is a photograph showing the inventive conformer containing 5% by weight loess.

FIG. 8 is a photograph showing the inventive conformer containing 20% by weight loess.

FIG. 9 is a photograph showing the inventive conformer containing 10% by weight bioceramic.

FIG. 10 is a photograph showing the inventive conformer containing 10% by weight zeolite.

FIG. 11 is a photograph showing the inventive conformer containing 10% by weight bentonite.

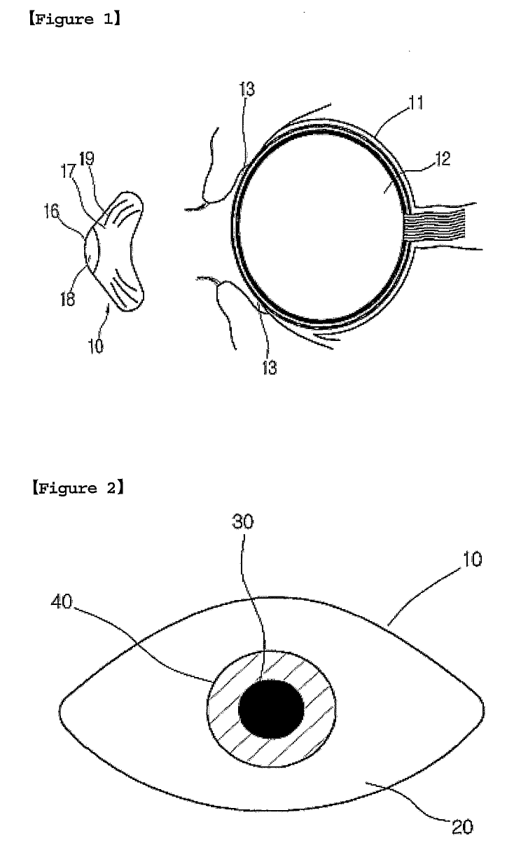

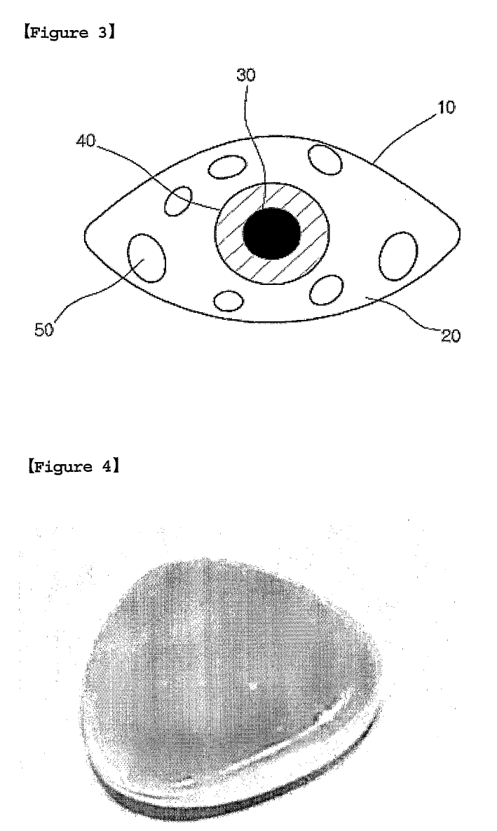

### BEST MODE

In one embodiment of producing an artificial eye with the addition of loess, the loess is mixed with transparent or non-transparent polymethylmethacrylate (PMMA) resin powder, and the mixed powder is mixed with PMMA resin liquid and gelled at room temperature.

In the mixing ratio, 1 g of loess is mixed with 10 g of the polymethylmethacrylate (PMMA) resin powder and then with 5 g of the PMMA resin solution. The mixing ratio can be adjusted according to the condition or request of a patient, and the acrylate resin powder can be used as a transparent or non-transparent material, as required.

When the content of loess is low, its effect will be insignificant, and when loess is used in an amount of more than 30% by weight, it will result in a deep color and decrease the beauty of a conformer.

The gelled mixture is placed and compressed in a plaster mole, and heated at 71-74° C. for 1 hour and 30 minutes and then at 100° C. for 30 minutes, thus making a complete artificial eye.

### Mode for Invention

In another embodiment, one corresponding to a portion of an artificial eye is prepared with the addition of loess, bentonite, zeolite, nano-silver, or bioceramic material. The prepared portion of an artificial eye is compressed together with the remaining portion of an artificial eye prepared without the addition of the above component, thus making one complete artificial eye.

In this case, a portion of the artificial eye, which is prepared with the addition of the above component, preferably forms the back side of the artificial eye in view of beauty when a person wears the artificial eye.

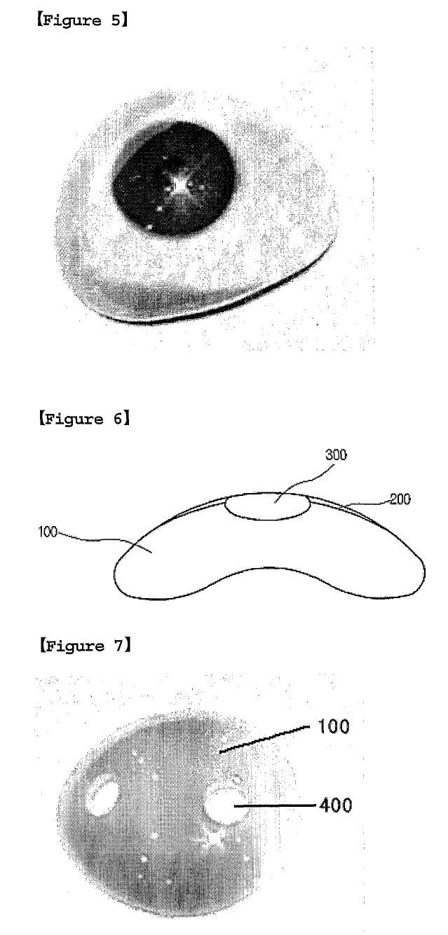

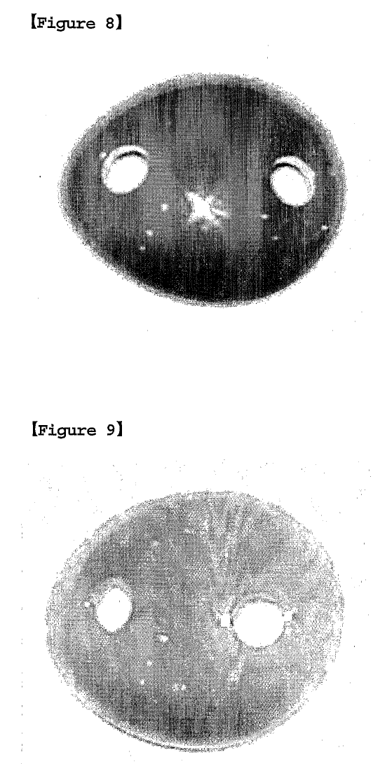

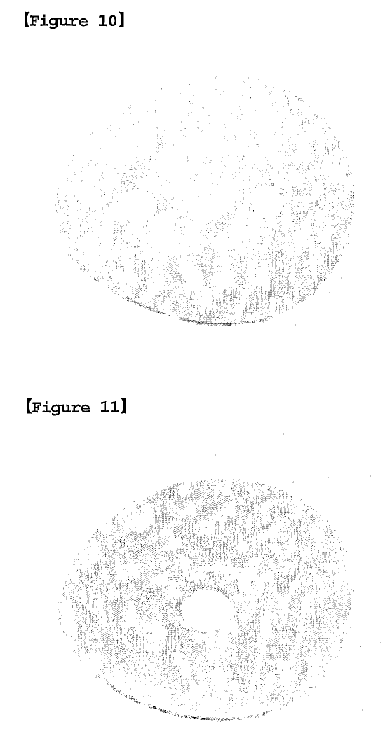

A photograph of the artificial eye containing loess only in a portion thereof as described above is shown in FIG. 4 showing the back side of the artificial eye, and a photograph of the artificial eye containing nano-silver only in a portion thereof is shown in FIG. 5 showing the front side of the artificial eye.

As a result, in producing the artificial eye with the addition of loess, bentonite, zeolite, nano-silver or bio-ceramic material, (1) this component may be added throughout the artificial eye, and (2) a portion of the artificial eye prepared with the addition of this component may be compressed together with the remaining artificial eye portion with no addition of this component.

When nano-silver is added to the artificial eye, it is preferably used in an amount of 0.01-5% by weight. This is because when too much nano-silver is used, it will result in a deep color, and when too little is used, it will have little or no effect and will injure the beauty of the artificial eye.

However, at least 0.001% by weight of nano-silver must be added in order to achieve the desired effect, and the amount used may vary according to the demand and request of a user.

Nano-silver powder or liquid is added in an amount of 0.01-5% by weight to plastic resin, acrylic resin, rubber or silicone, which is the material of the artificial eye.

A production process thereof is the same as the above-described process of producing the artificial eye with the addition of loess.

In another embodiment, an artificial eye is previously prepared, and then, in the final step, one or more selected from loess, bentonite, zeolite, nano-silver and bioceramic is coated or applied on the outer surface of the prepared artificial eye.

In still another embodiment, nano-gold or nano-platinum can be used to produce an artificial eye, in which case a method of producing the artificial eye with the addition of nano-gold or nano-platinum is the same as the production method with the addition of nano-silver.

When nano-gold or nano-platinum is added to produce an artificial eye, it is most preferably used in an amount of 0.01-5% by weight for the same reason as in the case of adding nano-silver.

However, 0.001% by weight of nano-gold or nano-platinum must be added in order to achieve the minimum desired effect, and the amount used can vary depending on the desire of a user.

### INDUSTRIAL APPLICABILITY

The present invention relates to an artificial eye and a conformer which is temporarily used by being inserted into an eye region after eyeball surgery. More particularly, the present invention relates to a bio-artificial eye and a bio-conformer, which are produced by a process comprising adding loess, zeolite, bentonite, bioceramic, nano-silver, nano-gold or nano-platinum to acrylic resin powder or silicone, so that the artificial eye and the conformer have antibacterial activity in themselves, do not cause much eye discharge, and emit far-infrared rays and anions, and thus, have a very good effect on the ocular health of a person wearing the artificial eye.

1. An artificial eye comprising polymethylmethacrylate (PMMA) resin and any one selected from the group consisting of loess, bentonite, zeolite and bioceramic.

2. The artificial eye of claim 1, wherein the content of any one of the loess, bentonite, zeolite and bioceramic components is less than 30% by weight, and the remaining component is the polymethylmethacrylate (PMMA) resin.

3. An artificial eye comprising nano-silver and polymethylmethacrylate (PMMA) resin.

4. The artificial eye of claim 3, wherein the content of the nano-silver component is 0.01-5% by weight, and the remaining component is the polymethylmethacrylate (PMMA) resin.

5. The artificial eye of claim 3, wherein the content of the nano-silver component is 0.001-5% by weight, and the remaining component is the polymethylmethacrylate (PMMA) resin.

6. An artificial eye comprising polymethylmethacrylate (PMMA) resin and any one of the nano-gold and nano-platinum components.

7. The artificial eye of claim 6, wherein the content of any one of the nano-gold and nano-platinum components is 0.01-5% by weight, and the remaining component is the polymethylmethacrylate (PMMA) resin.

8. The artificial eye of claim 6, wherein the content of any one of the nano-gold and nano-platinum components is 0.001-5% by weight, and the remaining component is the polymethylmethacrylate (PMMA) resin.

9. An artificial eye comprising silicone and any one selected from the group consisting of loess, bentonite, zeolite and bioceramic.

10. An artificial eye comprising silicone and any one selected from the group consisting of nano-silver, nano-gold and nano-platinum.

11. A conformer which is temporarily used by being inserted into an eye region after eyeball surgery, in which the conformer comprises acrylic resin powder and any one component selected from loess, bentonite, zeolite, bio-ceramic, and nano-silver.

12. A conformer which is temporarily used by being inserted into an eye region after eyeball surgery, in which the conformer comprises silicone and any one component selected from loess, bentonite, zeolite, bio-ceramic, and nano-silver.
